(19) United States
(12) Patent Application Publication
(10) Pub. No.: US 2025/0188468 A1
(43) Pub. Date: Jun. 12, 2025

(54) MICRORNAS FROM FORSYTHIAE FRUCTUS-ASTRAGALI RADIX COMPOUND TRADITIONAL CHINESE MEDICINE DECOCTION AS WELL AS PREPARATION METHOD AND USE THEREOF

(71) Applicant: NANTONG UNIVERSITY

(72) Inventors: Xiaosong GU, Jiangsu (CN); Susu MAO, Jiangsu (CN); Leilei GONG, Jiangsu (CN); Xiaoming YANG, Jiangsu (CN); Xinghui WANG, Jiangsu (CN); Hualin SUN, Jiangsu (CN); Lai XU, Jiangsu (CN)

(73) Assignee: NANTONG UNIVERSITY, Jiangsu (CN)

(21) Appl. No.: 18/279,209

(22) PCT Filed: May 25, 2022
(86) PCT No.: PCT/CN2022/094829

§ 371 Date: Sep. 2, 2024

(30) Foreign Application Priority Data
Apr. 21, 2022 (CN) 202210424718.8

(51) Int. Cl. C12N 15/113; A61K 31/7105; A61P 31/16; C12N 15/10

(52) U.S. Cl. CPC C12N 15/1131; A61K 31/7105; A61P 31/16; C12N 15/101; C12N 2310/141

(57) ABSTRACT

Provided are a Forsythia suspensa and Radix astragali compound traditional Chinese medicine decoction-derived micro ribonucleic acid and a preparation method therefor. The micro ribonucleic acid is selected from miRNAs with nucleotide sequences as shown in SEQ ID NOs. 1-15. Further provided are a miRNA QQ_159 with the nucleotide sequence as shown in SEQ ID NO. 14, comprising an artificially synthesized QQ_159, a plant QQ_159, a precursor form of the QQ_159, or a mature form of the QQ_159, use of the miRNA QQ_159 in the preparation of a drug for treating viral pneumonia caused by viral influenza and SARS-COV-2 virus, and a pharmaceutical composition comprising the miRNA QQ_159 and a pharmaceutically acceptable carrier.

### TECHNICAL FIELD

The present disclosure belongs to the technical field of biological medicines, and specifically relates to microRNAs derived from a Forsythiae Fructus-Astragali Radix compound traditional Chinese medicine decoction as well as a preparation method and a use thereof.

### BACKGROUND ART

Influenza (flu for short) is an acute respiratory disease commonly suffered by mammals and poultry, is caused by influenza virus, has strong infectivity and high transmission speed, generally causes simple respiratory infection including cough, fever, myalgia, chill or sweating and other clinical symptoms, which can keep uncomfortable feeling for 2 days to 8 days, and generally causes rapid disease. Some patients, especially the elderly, young children and patients with other chronic diseases, are prone to viral or secondary pneumonia, even accompanied by respiratory difficulties and multiple organ failure. Anti-influenza medication is a common approach for resisting influenza at present, and the prescription is generally determined according to the infection degree of a patient and the physical condition of the patient. Although influenza virus is one of the most deeply studied pathogens, the existing control and treatment schemes need to be continuously improved due to the extreme variability of influenza virus, and the development of new drugs for viral influenza is a great demand of both the current society and the civil health.

The novel coronavirus (nCOV for short) is a novel virus widely spread in recent two years, is mainly spread through a respiratory pathway in a droplet form, and recently, the novel coronavirus is further found to be spread through aerosol and article contact. The novel coronavirus has extremely strong infectivity and extremely high spreading speed, and clinical symptoms are mainly as shown in follows: fever, fatigue, dry cough, olfactory deficits/disorders, severe cases may be accompanied by severe pneumonia with dyspnea, tachypnea and hypoxemia and may cause other complications. Because there is no specific therapeutic drug aiming at the new coronavirus at present, the coronavirus can be spread on a large scale all over the world. Therefore, in view of the medicine requirements for epidemic prevention and control, researching and developing a new medicine specific to the nCOV is an important means for dealing with the nCOV and guaranteeing the life safety of people.

MicroRNAs (miRNAs for short) are a micro endogenous non-coding RNAs. Typically, the miRNA coding gene is transcribed by RNA polymerase II and produces a primary transcript, that is processed by RNase III endonuclease Drosha and Dicer into microRNAs of approximately 21 nucleotides. MiRNA mediates post-transcriptional gene silencing through the binding to coding regions or 3′ and 5′ non-translated regions of target mRNA, which plays an important role in various physiological and pathological processes, and has wide application prospects in the medical field.

In recent years, the related research of plant miRNA is rapidly developed, the biological function of the plant miRNA is mainly researched through an up-regulation/down-regulation strategy, and a plurality of researches show that the plant miRNA plays an important role in the aspects of photosynthesis, nutrition homeostasis, growth and development, hormone signal transduction, stress response and the like; in addition, plant miRNAs have been shown to regulate animal gene expression as endogenous mirnas. A great number of literatures report that traditional Chinese medicines play an active role in the process of preventing and treating influenza and novel coronavirus pneumonia. However, the specific active ingredient is still unclear. There have been literatures report that the traditional Chinese medicine contains miRNA, but there is still little understanding on the kind of stable miRNAs exist in traditional Chinese medicine decoction, and the functions these miRNAs will play when entering the animal bodies.

### SUMMARY OF THE INVENTION

In view of the technical deficiencies in the prior art, the present disclosure provides microRNAs derived from a Forsythiae Fructus-Astragali Radix compound traditional Chinese medicine decoction as well as a preparation method and a use thereof, functional plant microRNAs or extract containing the microRNAs extracted from the Forsythiae Fructus-Astragali Radix compound traditional Chinese medicine decoction, and proves through experimental testings that the microRNAs QQ_159 from the Forsythiae Fructus-Astragali Radix compound traditional Chinese medicine decoction has certain inhibiting effect and treating effect on viral influenza and pneumonia caused by novel coronavirus infection.

The present disclosure is achieved through the following technical solutions.

Provided is microRNAs derived from a Forsythiae Fructus-Astragali Radix compound traditional Chinese medicine decoction. The microRNAs are selected from novel_mir7, novel_mir33, novel_mir35, novel_mir10, novel_mir20, novel_mir5, novel_mir36, novel_mir28, novel_mir31, ppt-miR894, novel_mir32, novel_mir21, pab-miR3711. QQ_159 or bdi-miR159a-3p; wherein
a nucleotide sequence of the novel_mir7 is as shown in SEQ ID NO.1;
a nucleotide sequence of the novel_mir33 is as shown in SEQ ID NO.2;
a nucleotide sequence of the novel_mir35 is as shown in SEQ ID NO.3;
a nucleotide sequence of the novel_mir10 is as shown in SEQ ID NO.4:
a nucleotide sequence of the novel_mir20 is as shown in SEQ ID NO.5:
a nucleotide sequence of the novel_mir5 is as shown in SEQ ID NO.6;
a nucleotide sequence of the novel_mir36 is as shown in SEQ ID NO.7;
a nucleotide sequence of the novel_mir28 is as shown in SEQ ID NO.8;
a nucleotide sequence of the novel_mir31 is as shown in SEQ ID NO.9;
a nucleotide sequence of the ppt-miR894 is as shown in SEQ ID NO.10;
a nucleotide sequence of the novel_mir32 is as shown in SEQ ID NO.11;
a nucleotide sequence of the novel_mir21 is as shown in SEQ ID NO.12;
a nucleotide sequence of the pab-miR3711 is as shown in SEQ ID NO.13;
a nucleotide sequence of the QQ_159 is as shown in SEQ ID NO.14; and
a nucleotide sequence of the bdi-miR159a-3p is as shown in SEQ ID NO.15.

Preferably, the microRNAs are QQ_159, and comprises artificially synthesized QQ_159, plant QQ_159 and a precursor form of QQ_159 or a mature form of QQ_159.

A method for preparing the microRNAs derived from a Forsythiae Fructus-Astragali Radix compound traditional Chinese medicine decoction includes the following steps.

In step 1), 300 mL of the Forsythiae Fructus-Astragali Radix compound traditional Chinese medicine decoction are divided and filled respectively into 50 mL enzyme removing centrifuge tubes; impurities are removed by centrifugation at 2000 rpm; 10 mL of supernatant are sucked from each tube and placed on ice, 30 mL of TRIzol are added at the ratio of 3 times the volume of the decoction, kept at the room temperature for 10 minutes after shaking uniformly with force; 6 mL of chloroform are subsequently added, shaked uniformly with force, and kept for 10 minutes.

In Step 2), centrifugation is carried out at 10000 g and 4° C. for 10 minutes, and supernatant is collected.

In Step 3), isopropanol with the same volume as that of the mixture is added to precipitate for 1 hour at the temperature of 20° C., and then the precipitate is centrifuged at 12000 g for 15 minutes.

In Step 4), the supernatant is carefully discarded, 5 mL of 75% ethanol is added to wash the precipitate, and the precipitate is centrifuged at 12000 g for 5 minutes at 4° C.

In Step 5), after centrifugation, the ethanol is carefully removed, the precipitate is left, after the precipitate is dried, 200 μL of 65° C. DEPC-treated water is added for dissolving, and the RNA concentration is determined.

In Step 6), the miRNAs are purified with a commercial miRNA isolation kit, 100 μg of RNAs pass through each column, and the miRNAs in each tube are dissolved by 60 μL of DEPC-treated water.

In Step7), the concentration of miRNAs is determined, freeze-dried for 8 hours, and stored at −80° C.

Provided is a use of the microRNAs QQ_159 derived from the Forsythiae Fructus-Astragali Radix compound traditional Chinese medicine decoction in a preparation of a medicament for inhibiting the replication of influenza virus.

Provided is a use of the microRNAs QQ_159 derived from the Forsythiae Fructus-Astragali Radix compound traditional Chinese medicine decoction in a preparation of a medicament for treating viral influenza.

Preferably, the influenza comprises Victoria influenza B or H5N1avian influenza.

Provided is a use of the microRNAs QQ_159 derived from the Forsythiae Fructus-Astragali Radix compound traditional Chinese medicine decoction in a preparation of a medicament for inhibiting the replication of SARS-COV-2 virus.

Provided is a use of the microRNAs QQ_159 derived from the Forsythiae Fructus-Astragali Radix compound traditional Chinese medicine decoction in a preparation of a medicament for treating viral pneumonia caused by SARS-COV-2 virus.

A pharmaceutical composition for inhibiting influenza virus replication and/or treating influenza includes the microRNAs QQ_159, and a pharmaceutically acceptable carrier thereof.

A pharmaceutical composition for inhibiting the replication of SARS-COV-2 virus and/or treating viral pneumonia caused by SARS-COV-2 virus includes the microRNAs QQ_159, and a pharmaceutically acceptable carrier thereof.

Provided is a method for inhibiting replication of Victoria B influenza virus for non-therapeutic purposes in vitro, the above-mentioned microRNA QQ_159 is contacted with a cell infected with influenza B influenza virus.

The present disclosure has the following beneficial effects.

The present disclosure provides an experimental testing method for extracting microRNAs from a traditional Chinese medicine decoction, and 15 kinds of microRNAs stably exist in a Forsythiae Fructus-Astragali Radix compound traditional Chinese medicine decoction are extracted and identified by the method for the first time. It is demonstrated that the microRNAs QQ_159 have a certain inhibitory effect and therapeutic effect on pneumonia caused by viral influenza and novel coronavirus infection. The present disclosure not only obtains natural antiviral effective components of the traditional Chinese medicine, but also provides a possible nucleic acid medicine for clinically treating influenza or pneumonia caused by novel coronavirus infection.

### BRIEF DESCRIPTION OF THE DRAWINGS

FIG. 1 illustrates the inhibition effect of the microRNAs QQ_159 on the influenza B virus load tested by the real-time PCR in Example 2.

FIG. 2 illustrates the effect of the microRNAs QQ_159 on body weight of mice infected with avian influenza H5N1 virus in Example 3.

FIG. 3 illustrates the effect of the microRNAs QQ_159 on the survival rate of mice infected with avian influenza H5N1 virus in Example 3.

FIG. 4 illustrates the inhibition effect of the microRNAs QQ_159 on lung inflammation in mice infected with avian influenza H5N1 through the HE staining test in Example 3.

FIG. 5 illustrates the inhibition effect of the microRNAs QQ_159 on the pulmonary viral load of mice infected with avian influenza H5N1 on days 3 (A) and days 5 (B) tested by the real-time PCR in Example 3.

FIG. 6 illustrates the inhibition effect of the microRNAs QQ_159 on lung inflammation in mice infected with SARS-COV-2 virus through the HE staining detection in Example 4.

### DETAILED DESCRIPTION OF THE EMBODIMENTS

In order to make the technical problems, technical solutions and advantages of the present disclosure more distinct, the following detailed description and specific embodiments are described with reference to the accompanying drawings.

### Example 1: Extraction of microRNAs from a Forsythiae Fructus-Astragali Radix Compound Traditional Chinese Medicine Decoction

(1) 300 mL of a Forsythiae Fructus-Astragali Radix compound traditional Chinese medicine (Lonicerae Japonicae Flos, Forsythiae Fructus, Scutellariae Radix, Artemisia Annuae Herba, raw Astragali Radix, roasted Atractylodis Macrocephalae Rhizoma, Agastache rugosa, Saposhnikoviae Radix, Ophiopogon japonicus, Glycyrrhiza uralensis) decoction are respectively divided and filled into 50 mL enzyme-removing centrifuge tubes, and impurities are removed by centrifugation at 2000 rpm; 10 mL of supernatant are sucked from each tube and placed on ice; 30 mL of TRIzol are added at the ratio of 3 times the volume of the decoction, kept at the room temperature for 10 minutes after shaking uniformly with force, 6 mL of chloroform are subsequently added, shaked uniformly with force, and kept for 10 minutes.
(2) Centrifugation is carried out at 10000 g and 4° C. for 10 minutes, and the supernatant is collected.
(3) Isopropanol with the same volume as that of the mixture is added to precipitate for 1 hour (−20° C.), and then the precipitate is centrifuged at 12000 g for 15 minutes.
(4) The supernatant is carefully discarded, 5 mL of 75% ethanol is added to wash the precipitate, and the precipitate is centrifuged at 12000 g for 5 minutes at 4° C.
(5) After centrifugation, the ethanol is carefully removed, the precipitate is left, after the precipitate is dried, and 200 μL of 65° C. DEPC-treated water is added to dissolve the precipitate, and the RNA concentration is determined.
(6) The miRNAs are purified by a commercial miRNA isolation kit (100. μg of RNAs pass through each column) and dissolved in each tube by 60 μL DEPC-treated water.
(7) The concentration of miRNAs is determined, freeze-dried for 8 hours, and stored at −80° C.
(8) The miRNAs are sequenced by BGISEQ-500 technique, and the results are as shown in Table 1 below.

TABLE 1
Sequence list of microRNAs derived from a
Forsythiae Fructus-Astragali Radix compound
traditional Chinese medicine decoction

| | SEQ | |
|---|---|---|
| miRNA id | ID | Sequence (5′-3′) |
| novel_mir7 | 1 | CCAGCACUAAUGCACCGGAUCCCAUCAG |
| novel_mir33 | 2 | UGGGAAGUCCUCGUGUUGUACCCCU |
| novel_mir35 | 3 | GCCUUCGAUGUCGGCUCUUCCUAUCAUU |
| novel_mir10 | 4 | UCGUCCAGCGGUUAGGAUAUCUGGCUUUC |
| novel_mir20 | 5 | GCGGAUCUUGGUGGUAGUAGCAAA |
| novel_mir5 | 6 | CUCGGGAAAAGGAUUGGCUCUGAGGGCUGG |
| novel_mir36 | 7 | ACACAUGCAAGUCGAACGUUGUUUU |
| novel_mir28 | 8 | GUGUGCACCGGUCGUCUCGUCCCUUCUG |
| novel_mir31 | 9 | UCUGGGUGGUGUAGUUGGUUAUCA |
| ppt-miR894 | 10 | CGUUUCACGUCGGGUUCACC |
| novel_mir32 | 11 | GUGGUUAGGACAUCGUCUUUUCA |
| novel_mir21 | 12 | GUCUGUAGUUCGAUCCUGCAUGGGGG |
| pab-miR3711 | 13 | UGGCGCUAGAAGGAGGGCCU |
| QQ_159 | 14 | UUUGGAUUGAAGGGAGCUCUA |
| bdi- | 15 | CUUGGAUUGAAGGGAGCUCU |
| miR159a-3p | | |

### Example 2: Determination of the Effect of QQ_159 on Influenza B Virus Load in Madin Darby Dog Kidney (MDCK) Cells

(1) MDCK cells are cultured in 24-well culture plates.
(2) 10 nM, 50 nM or 100 nM QQ_159a are transfected into MDCK cells respectively by the commercial transfection reagent riboFECTTM CP, while the nonsense sequence with the same concentration is transfected into MDCK cells as a control.
(3) The above-mentioned cells are infected with Victoria B influenza virus.
(4) After infecting the virus for 24 hours, the supernatant is collected and centrifuged, and cells are merged, RNAs are extracted, and the virus load is detected by real-time PCR.

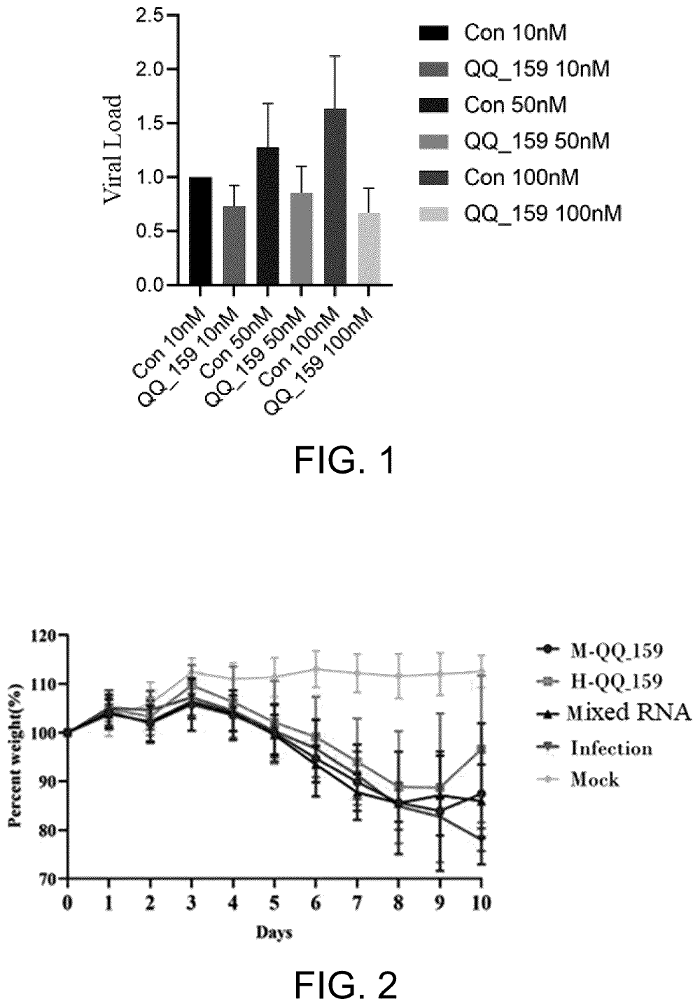

The results are as illustrated in FIG. 1, and 100 nM QQ_159 can significantly inhibit the viral load of Victoria B influenza virus in MDCK cells compared with the control group.

### Example 3 Determination of the Therapeutic Effect of QQ_159 on Mice Infected with H5N1 Subtype Avian Influenza Virus

(1) Medicine are injected intraperitoneally to 6-week-old BALB/C female mice 2 hours after infected with H5N1 subtype avian virus. The animal groups and the drug dosages are as shown in Table 2 below.

TABLE 2
QQ_159 avian influenza resistant H5N1 animal experimental testing groups
and medicine treatment

| | | Dosages of | Administration | Administration | Administration | Number |
|---|---|---|---|---|---|---|
| Groups | | virus attack | dosages | mode | times | of mice |
| Group | M- | 30 | 120 pmol/per | Intraperitoneal | 5 | 16 |
| 1 | QQ_159 | LD50/50 μL | mouse/day | injection | | |
| | | | 0.1 mL | (DEPC-treated | | |
| | | | | water dilution | | |
| | | | | QQ_159) | | |
| Group | H- | 30 | 360 pmol/per | Intraperitoneal | 5 | 16 |
| 2 | QQ_159 | LD50/50 μL | mouse/day | injection | | |
| | | | 0.1 mL | (DEPC-treated | | |
| | | | | water dilution | | |
| | | | | QQ_159) | | |
| Group | Mixed | 30 | 270 ug/per | Intraperitoneal | 5 | 16 |
| 3 | RNAGr | LD50/50 μL | mouse/day | injection | | |
| | oup | | 0.1 mL | (DEPC-treated | | |
| | | | | water dilution | | |
| | | | | mixed RNA) | | |
| Group | Infection | 30 | DEPC-treated | Intraperitoneal | 5 | 16 |
| 4 | Control | LD50/50 μL | water | injection | | |
| | Group | | 0.1 mL | (DEPC-treated | | |
| | | | | water) | | |
| Group | Normal | DEPC- | DEPC-treated | Intraperitoneal | 5 | 16 |
| 5 | control | treated | water | injection | | |
| | group | water 50 μL | 0.1 mL | (DEPC-treated | | |
| | | | | water) | | |

In Table 2: the mixed RNAs are mixed RNAs extracted from a Forsythiae Fructus-Astragali Radix compound traditional Chinese medicine decoction in a gathering way.
(2) The administration is continued for 5 days, and the change in body weights is observed and recorded every day (the mice are euthanized as death when the body weights are decreased by more than 30%).
(3) Lung tissues are taken on the third and fifth days after the virus attack respectively, and the inflammation condition of the lung tissues is detected by HE staining.
(4) Lung tissue viral load is detected through real-time PCR.
(5) Animals are housed until day 10 and the survival rate is observed.

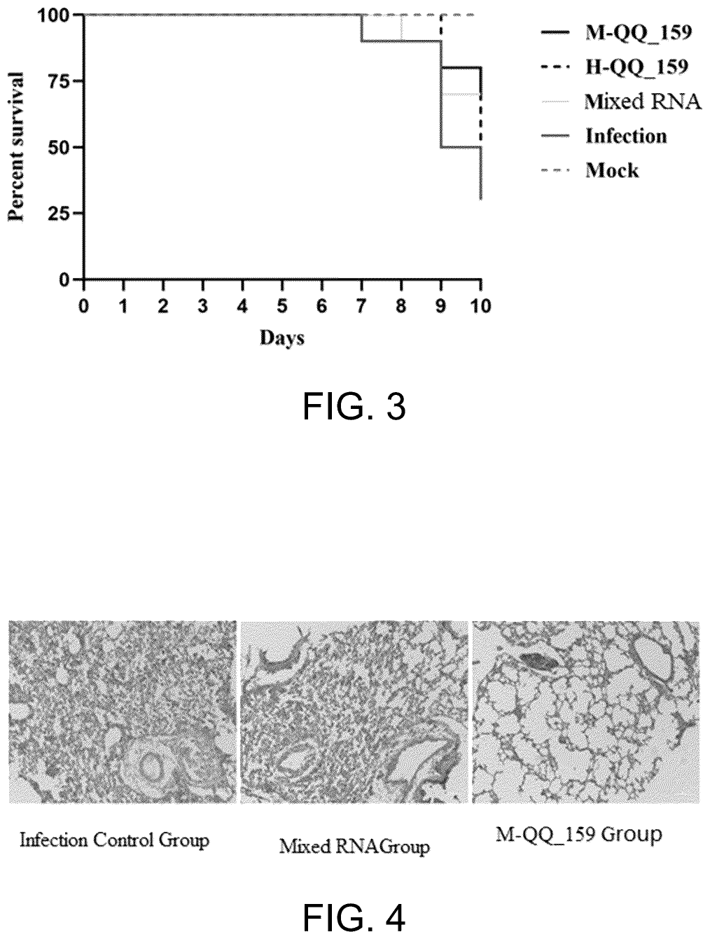

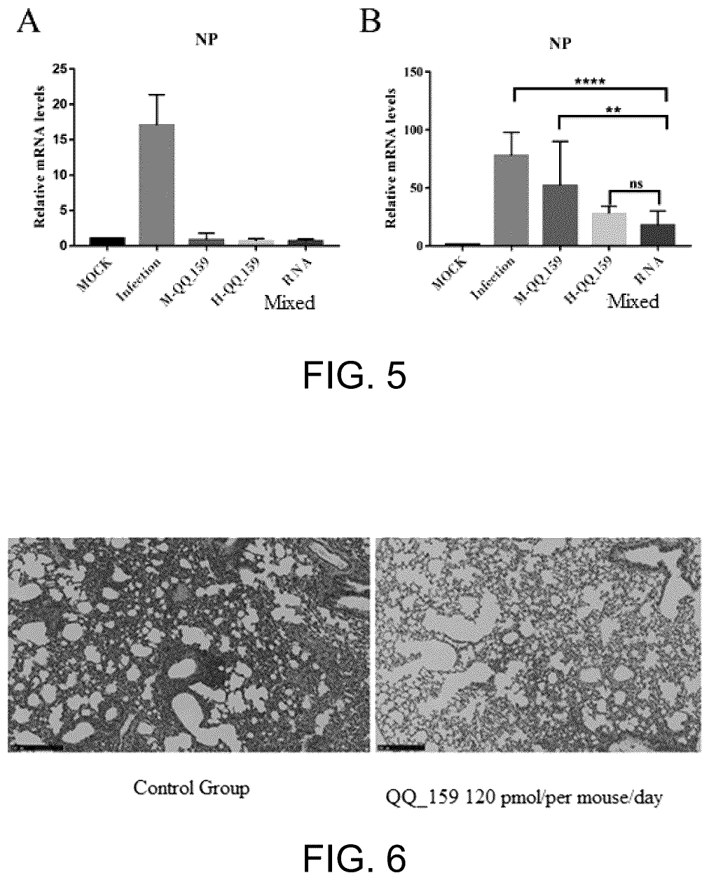

The result is that: in terms of change of the body weight, as illustrated in FIG. 2, the body weight changes of medium concentration QQ_159 (120 pmol/per mouse/day, M-QQ_159), high concentration QQ_159 (360 pmol/per mouse/day, H-QQ_159), and mixed RNA group at 7 days after virus attack (D8 is illustrated in FIG. 2) enter plateau, but the infected control group still have a tendency to decrease; the rate of change in body weight is slightly higher in the high concentration QQ_159 group than those in the mixed RNA group and the medium concentration QQ_159. In terms of survival rate, as illustrated in FIG. 3, the survival rate of the medium-concentration QQ_159 group and the mixed RNA group is 70%, the survival rate of the high-concentration QQ_159 group is 50%, and the survival rate of the infected group is merely 30%. As illustrated in FIG. 4, HE results show that lung inflammation is significantly reduced in the mixed RNA group and medium-concentration QQ_159 mice, and better inhibitory effect is exhibited in the medium-concentration QQ_159 than the infection control group. As illustrated in A of FIG. 5, the medium-concentration QQ_159 group, the high-concentration QQ_159 group, and the mixed RNA group are all effective in inhibiting viral replication on the third day after infection; as illustrated in B of FIG. 5, on the fifth day after infection, the medium concentration QQ 159 group exhibits a significantly reduced effect of inhibiting viral replication, while the high concentration QQ_159 group and the mixed RNA group still exhibit a better effect of inhibiting viral replication.

The experimental testing shows that the artificially synthesized QQ_159 and the mixed RNA extracted from a Forsythiae Fructus-Astragali Radix compound traditional Chinese medicine decoction have certain treatment effect on the H5N1 avian influenza virus, which is represented by delaying weight loss, reducing mortality and reducing lung inflammation.

### Example 4 Research of the Inhibition Effect of QQ_159 on Pneumonia Caused by SARS-CoV-2 Virus

(1) The hACE2 mice at the age of 6 to 8 weeks are infected with SARS-COV-2 at 10⁵TCID₅₀/ml in form of nasal drops.
(2) Mice are administered by intraperitoneal (ip) injection for 5 days. The mice are administered after being infected for 2 hours on the first day. The experimental testing groups and the dosages are as shown in Table 3 below.

TABLE 3
SARS-CoV-2 infection experimental testing of hACE2 transgenic mice

| | Administration | Administration | Administration | Time of virus | |
|---|---|---|---|---|---|
| Group | dosages | volume | mode | attack | Times |
| 1 | 60 pmol/per | 100 μL/per | ip | Administration | 5 |
| | mouse/day | mouse | | after infection | |
| | | | | of 2 h | |
| 2 | 120 pmol/per | 100 μL/per | ip | Administration | 5 |
| | mouse /day | mouse | | after infection | |
| | | | | of 2 h | |
| 3 | 180 pmol/per | 100 μL/per | ip | Administration | 5 |
| | mouse /day | mouse | | after infection | |
| | | | | of 2 h | |
| Control | — | 100 μL/per | ip | Virus attack at | 5 |
| Group | | mouse | | day 0 | |

(3) Body weight and symptoms are monitored on days 0, 1, 2, 3, 4, 5.
(4) Mice are sacrificed on the fifth day after infection and lung tissues are collected.
(5) Lung pathology is detected by HE staining

As illustrated in FIG. 6, 120 pmol/per mouse/day injection of QQ_159 exhibits a certain inhibitory effect on the lung tissue inflammation of hACE2 transgenic mice infected with SARS-CoV-2, indicating that QQ_159 exhibits a certain inhibitory effect on the mouse pneumonia caused by SARS-COV-2 infection.

The above are merely preferred embodiments of the present disclosure, and it should be noted that for those skilled in the art, a plurality of modifications and adaptations can be made without departing from the principle of the present disclosure, and these modifications and adaptations should be considered to be within the protection scope of the present disclosure.

1. MicroRNAs derived from a Forsythiae Fructus-Astragali Radix compound traditional Chinese medicine decoction, wherein the microRNAs are selected from novel_mir7, novel_mir33, novel_mir35, novel_mir10, novel_mir20, novel_mir5, novel_mir36, novel_mir28, novel_mir31, ppt-miR894, novel_mir32, novel_mir21, pab-miR3711, QQ_159 or bdi-miR159a-3p; wherein
a nucleotide sequence of the novel_mir7 is as shown in SEQ ID NO.1;
a nucleotide sequence of the novel_mir33 is as shown in SEQ ID NO.2;
a nucleotide sequence of the novel_mir35 is as shown in SEQ ID NO.3;
a nucleotide sequence of the novel_mir10 is as shown in SEQ ID NO.4;
a nucleotide sequence of the novel_mir20 is as shown in SEQ ID NO.5;
a nucleotide sequence of the novel_mir5 is as shown in SEQ ID NO.6;
a nucleotide sequence of the novel_mir36 is as shown in SEQ ID NO.7;
a nucleotide sequence of the novel_mir28 is as shown in SEQ ID NO.8;
a nucleotide sequence of the novel_mir31 is as shown in SEQ ID NO.9;
a nucleotide sequence of the ppt-miR894 is as shown in SEQ ID NO.10;
a nucleotide sequence of the novel_mir32 is as shown in SEQ ID NO.11;
a nucleotide sequence of the novel_mir21 is as shown in SEQ ID NO.12;
a nucleotide sequence of the pab-miR3711 is as shown in SEQ ID NO.13;
a nucleotide sequence of the QQ_159 is as shown in SEQ ID NO.14; and
a nucleotide sequence of the bdi-miR159a-3p is as shown in SEQ ID NO.15.

2. The microRNAs derived from the Forsythiae Fructus-Astragali Radix compound traditional Chinese medicine decoction according to claim 1, wherein the microRNAs are QQ_159, and comprise artificially synthesized QQ_159, plant QQ_159 and a precursor form of QQ_159 or a mature form of QQ_159.

3. A method for preparing the microRNAs derived from the Forsythiae Fructus-Astragali Radix compound traditional Chinese medicine decoction according to claim 1, wherein the method comprises following steps:
Step 1), respectively dividing and filling 300 mL of the Forsythiae Fructus-Astragali Radix compound traditional Chinese medicine decoction into 50 mL enzyme removing centrifuge tubes, and centrifuging at 2000 rpm to remove impurities; sucking 10 mL of supernatant from each tube and placing the supernatant on ice, adding 30 mL of TRIzolls at a ratio of 3 times a volume of the decoction, shaking uniformly with force, keeping at a room temperature for 10 minutes, subsequently adding 6 mL of chloroform, shaking uniformly with force, and keeping for 10 minutes;
Step 2), centrifuging at 10000 g and 4° C. for 10 minutes, and collecting the supernatant;
Step 3), adding isopropanol with a same volume as that of the supernatant to precipitate for 1 hour at a temperature of 20° C., and then centrifuging at 12000 g for 15 minutes;
Step 4), carefully discarding the supernatant, adding 5 mL of 75% ethanol to wash the precipitate, and centrifuging at 12000 g for 5 minutes at 4° C.;
Step 5), after centrifugation, carefully removing ethanol, leaving the precipitate, after drying the precipitate, adding 200 μL of 65° C. DEPC-treated water for dissolving, and determining a RNA concentration;
Step 6), purifying miRNAs with a commercial miRNA isolation kit, passing 100 μg of RNAs through each column, and dissolving the miRNAs in each tube by 60 μL of DEPC-treated water; and
Step7) determining a concentration of miRNAs, freeze-drying for 8 hours, and storing at −80° C.

4. Use of the microRNAs derived from the Forsythiae Fructus-Astragali Radix compound traditional Chinese medicine decoction according to claim 2 in a preparation of a medicament for inhibiting a replication of influenza virus.

5. Use of the microRNAs derived from the Forsythiae Fructus-Astragali Radix compound traditional Chinese medicine decoction according to claim 2 in a preparation of a medicament for treating viral influenza.

6. The use according to claim 4, wherein the influenza comprises Victoria influenza B or H5N1 avian influenza.

7. Use of the microRNAs derived from the Forsythiae Fructus-Astragali Radix compound traditional Chinese medicine decoction according to claim 2 in a preparation of a medicament for inhibiting a replication of SARS-COV-2 virus.

8. Use of the microRNAs derived from the Forsythiae Fructus-Astragali Radix compound traditional Chinese medicine decoction according to claim 2 in a preparation of a medicament for treating viral pneumonia caused by SARS-COV-2 virus.

9. A pharmaceutical composition for inhibiting influenza virus replication and/or treating influenza, comprising the microRNAs QQ_159 according to claim 2, and a pharmaceutically acceptable carrier thereof.

10. A pharmaceutical composition for inhibiting the replication of SARS-COV-2 virus and/or treating viral pneumonia caused by SARS-COV-2 virus, comprising the microRNAs QQ_159 according to claim 2, and a pharmaceutically acceptable carrier thereof.

11. A method for preparing the microRNAs derived from the Forsythiae Fructus-Astragali Radix compound traditional Chinese medicine decoction according to claim 2, wherein the method comprises following steps:
Step 1), respectively dividing and filling 300 mL of the Forsythiae Fructus-Astragali Radix compound traditional Chinese medicine decoction into 50 mL enzyme removing centrifuge tubes, and centrifuging at 2000 rpm to remove impurities; sucking 10 mL of supernatant from each tube and placing the supernatant on ice, adding 30 mL of TRIzolls at a ratio of 3 times a volume of the decoction, shaking uniformly with force, keeping at a room temperature for 10 minutes, subsequently adding 6 mL of chloroform, shaking uniformly with force, and keeping for 10 minutes;
Step 2), centrifuging at 10000 g and 4° C. for 10 minutes, and collecting the supernatant;
Step 3), adding isopropanol with a same volume as that of the supernatant to precipitate for 1 hour at a temperature of 20° C., and then centrifuging at 12000 g for 15 minutes;
Step 4), carefully discarding the supernatant, adding 5 mL of 75% ethanol to wash the precipitate, and centrifuging at 12000 g for 5 minutes at 4° C.;
Step 5), after centrifugation, carefully removing ethanol, leaving the precipitate, after drying the precipitate, adding 200 μL of 65° C. DEPC-treated water for dissolving, and determining a RNA concentration;
Step 6), purifying miRNAs with a commercial miRNA isolation kit, passing 100 μg of RNAs through each column, and dissolving the miRNAs in each tube by 60 μL of DEPC-treated water; and
Step7) determining a concentration of miRNAs, freeze-drying for 8 hours, and storing at −80° C.

12. The use according to claim 5, wherein the influenza comprises Victoria influenza B or H5N1 avian influenza.


SEQUENCE LISTING (15 sequences)

SEQ ID NO: 1 - RNA, 28 nt, Artificial Sequence
  Obtained from plants
ccagcacuaaugcaccggaucccaucag

SEQ ID NO: 2 - RNA, 25 nt, Artificial Sequence
  Obtained from plants
ugggaaguccucguguuguaccccu

SEQ ID NO: 3 - RNA, 28 nt, Artificial Sequence
  Obtained from plants
gccuucgaugucggcucuuccuaucauu

SEQ ID NO: 4 - RNA, 29 nt, Artificial Sequence
  Obtained from plants
ucguccagcgguuaggauaucuggcuuuc

SEQ ID NO: 5 - RNA, 24 nt, Artificial Sequence
  Obtained from plants
gcggaucuuggugguaguagcaaa

SEQ ID NO: 6 - RNA, 30 nt, Artificial Sequence
  Obtained from plants
cucgggaaaaggauuggcucugagggcugg

SEQ ID NO: 7 - RNA, 25 nt, Artificial Sequence
  Obtained from plants
acacaugcaagucgaacguuguuuu

SEQ ID NO: 8 - RNA, 28 nt, Artificial Sequence
  Obtained from plants
gugugcaccggucgucucgucccuucug

SEQ ID NO: 9 - RNA, 24 nt, Artificial Sequence
  Obtained from plants
ucugggugguguaguugguuauca

SEQ ID NO: 10 - RNA, 20 nt, Artificial Sequence
  Obtained from plants
cguuucacgucggguucacc

SEQ ID NO: 11 - RNA, 23 nt, Artificial Sequence
  Obtained from plants
gugguuaggacaucgucuuuuca

SEQ ID NO: 12 - RNA, 26 nt, Artificial Sequence
  Obtained from plants
gucuguaguucgauccugcauggggg

SEQ ID NO: 13 - RNA, 20 nt, Artificial Sequence
  Obtained from plants
uggcgcuagaaggagggccu

SEQ ID NO: 14 - RNA, 21 nt, Artificial Sequence
  Obtained from plants
uuuggauugaagggagcucua

SEQ ID NO: 15 - RNA, 20 nt, Artificial Sequence
  Obtained from plants
cuuggauugaagggagcucu